(19) United States
(12) Patent Application Publication
(10) Pub. No.: US 2025/0336540 A1
(43) Pub. Date: Oct. 30, 2025

(54) METABOLITE PREDICTORS FOR LUNG CANCER

(71) Applicant: Janssen Pharmaceutica NV, Beerse (BE)

(72) Inventors: Nastaran Emaminejad, Rancho Mission Viejo, CA (US); Takahiro Sato, Philadelphia, PA (US); Robert Yunchuan Yang, Belmont, MA (US); Jacobus Maurits Klap, Gouda (NL); Stefan Kostense, Voorburg (NL)

(21) Appl. No.: 18/860,730

(22) PCT Filed: Apr. 28, 2023
(86) PCT No.: PCT/EP2023/061371

§ 371 Date: Oct. 28, 2024

Related U.S. Application Data
(60) Provisional application No. 63/335,997, filed on Apr. 28, 2022.

(51) Int. Cl. G16H 50/30; G01N 33/574

(52) U.S. Cl. CPC G16H 50/30; G01N 33/57423

(57) ABSTRACT

Disclosed herein are methods for analyzing predictors including quantitative values of biomarkers (e.g., metabolite biomarkers) for predicting risk of cancer in a human subject. Further disclosed herein are kits for measuring quantitative values of the markers as well as computer systems and software embodiments for predicting risk of cancer in a human subject based on the quantitative values of the biomarkers (e.g., metabolite biomarkers).

### FIELD

The field relates to predictive models that are useful for predicting risk of cancer (e.g., lung cancer). These predictive models are based at least on the measurement of metabolite profiles from samples (e.g., peripheral blood plasma samples).

### BACKGROUND

Lung cancer is the leading cause of cancer deaths worldwide. This is largely due to its advanced stage at the time of diagnosis, with 5-year survival of only 15% or less. It is difficult to identify people who have early stage lung cancer in a cost-efficient manner. Hence, people are often referred to hospital clinics with late stage disease, which leads to poor curative opportunities and outlook.

### SUMMARY

Disclosed herein are methods for predicting risk of cancer (e.g., future risk of cancer or presence or absence of cancer) in a subject using multivariate panels, such as multivariate panels comprised of metabolite biomarkers. Additionally disclosed herein are non-transitory computer readable mediums for predicting risk of cancer in a subject using multivariate panels. Additionally disclosed herein are kits containing one or more sets of reagents for determining quantitative values of predictors for predicting risk of cancer. In various embodiments, the prediction for risk of cancer for the subject is a prediction of presence or absence of cancer in the subject, or a prediction of whether the subject is likely to develop cancer in the future (e.g., within 1-20 years). In various embodiments, the terms “levels” and “values”, such as the levels or values of metabolites, biomarkers, markers or predictors, are synonymous and may be used interchangeably. Therefore, in these embodiments, any reference to “values”, such as the values of metabolites, biomarkers, markers or predictors, may equally be construed as “levels”, such as the levels of those metabolites, biomarkers, markers or predictors. Similarly, in these embodiments, any reference to “levels”, such as the levels of metabolites, biomarkers, markers or predictors, may equally be construed as “values”, such as the values of those metabolites, biomarkers, markers or predictors.

Disclosed herein is a method for predicting risk of cancer in a subject, the method comprising: obtaining or having obtained a dataset comprising quantitative levels of a plurality of biomarkers, wherein the plurality of biomarkers comprises metabolite biomarkers comprising two or more of Beta-hydroxyisovaleroylcarnitine, Pyrraline, Citramalate, Succinate, and Urate, and generating a prediction of risk of cancer for the subject by applying a predictive model to the quantitative values of the plurality of biomarkers.

In various embodiments, the metabolite biomarkers comprise three or more of Beta-hydroxyisovaleroylcarnitine, Pyrraline, Citramalate, Succinate, and Urate. In various embodiments, the metabolite biomarkers comprise four or more of Beta-hydroxyisovaleroylcarnitine, Pyrraline, Citramalate, Succinate, and Urate. In various embodiments, the metabolite biomarkers comprise each of Beta-hydroxyisovaleroylcarnitine, Pyrraline, Citramalate, Succinate, and Urate. In various embodiments, the metabolite biomarkers further comprise one or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise five or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise ten or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise each of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

In various embodiments, the metabolite biomarkers further comprise one or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, the metabolite biomarkers further comprise five or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, the metabolite biomarkers further comprise ten or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, the metabolite biomarkers further comprise each of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

Additionally disclosed herein is a method for predicting risk of cancer in a subject, the method comprising: obtaining or having obtained a dataset comprising quantitative levels of a plurality of biomarkers, wherein the plurality of biomarkers comprises metabolite biomarkers comprising two or more of pseudoephedrine, 3-(cystein-S-yl) acetaminophen, 2-methoxyacetaminophen sulfate, alliin, and daidzein sulfate, and generating a prediction of risk of cancer for the subject by applying a predictive model to the quantitative values of the plurality of biomarkers. In various embodiments, the metabolite biomarkers comprise three or more of pseudoephedrine, 3-(cystein-S-yl) acetaminophen, 2-methoxyacetaminophen sulfate, alliin, and daidzein sulfate. In various embodiments, the metabolite biomarkers comprise four or more of pseudoephedrine, 3-(cystein-S-yl) acetaminophen, 2-methoxyacetaminophen sulfate, alliin, and daidzein sulfate. In various embodiments, the metabolite biomarkers comprise each of pseudoephedrine, 3-(cystein-S-yl) acetaminophen, 2-methoxyacetaminophen sulfate, alliin, and daidzein sulfate. In various embodiments, the metabolite biomarkers further comprise one or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, the metabolite biomarkers further comprise five or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, the metabolite biomarkers further comprise ten or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, the metabolite biomarkers further comprise each of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

In various embodiments, the metabolite biomarkers further comprise one or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In various embodiments, the metabolite biomarkers further comprise five or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In various embodiments, the metabolite biomarkers further comprise ten or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In various embodiments, the metabolite biomarkers further comprise each of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3 alpha, 17beta-diol monosulfate, and homocitrulline.

In various embodiments, the cancer is lung cancer. In various embodiments, the risk of cancer is a level of risk of the subject developing cancer within 1 year, within 2 years, within 3 years, within 4 years, within 5 years, within 6 years, within 7 years, within 8 years, within 9 years, or within 10 years. In various embodiments, the risk of cancer is a presence or absence of cancer. In various embodiments, the level of risk is one of a low risk, medium risk, or high risk. In various embodiments, the dataset is derived from a test sample obtained from the subject. In various embodiments, the test sample is a blood or serum sample. In various embodiments, obtaining or having obtained the dataset comprises performing one or more assays. In various embodiments, performing the one or more assays comprises performing one or more of liquid chromatography (LC), gas chromatography (GC) (e.g., GC using an electron capture detector), a nitrogen/phosphorous detector, a flame photometric detector, high performance liquid chromatography (HPLC), nuclear magnetic resonance (NMR), mass spectrometry (MS), liquid chromatography MS (LC-MS), high performance LC-MS (HPLC-MS), or ultrahigh performance liquid chromatography-tandem MS (UPLC-MS/MS). In various embodiments, methods disclosed herein further comprise: selecting a therapy for providing to the subject based on the prediction of cancer.

Additionally disclosed herein is a non-transitory computer readable medium comprising instructions that, when executed by a processor, cause the processor to: obtain or have obtained a dataset comprising quantitative levels of a plurality of biomarkers, wherein the plurality of biomarkers comprises metabolite biomarkers comprising two or more of Beta-hydroxyisovaleroylcarnitine, Pyrraline, Citramalate, Succinate, and Urate, and generate a prediction of risk of cancer for the subject by applying a predictive model to the quantitative values of the plurality of biomarkers.

In various embodiments, the metabolite biomarkers comprise three or more of Beta-hydroxyisovaleroylcarnitine, Pyrraline, Citramalate, Succinate, and Urate. In various embodiments, the metabolite biomarkers comprise four or more of Beta-hydroxyisovaleroylcarnitine, Pyrraline, Citramalate, Succinate, and Urate. In various embodiments, the metabolite biomarkers comprise each of Beta-hydroxyisovaleroylcarnitine, Pyrraline, Citramalate, Succinate, and Urate. In various embodiments, the metabolite biomarkers further comprise one or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise five or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise ten or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise each of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In various embodiments, the metabolite biomarkers further comprise one or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, the metabolite biomarkers further comprise five or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, the metabolite biomarkers further comprise ten or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, the metabolite biomarkers further comprise each of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

Additionally disclosed herein is a non-transitory computer readable medium comprising instructions that, when executed by a processor, cause the processor to: obtain or have obtained a dataset comprising quantitative levels of a plurality of biomarkers, wherein the plurality of biomarkers comprises metabolite biomarkers comprising two or more of pseudoephedrine, 3-(cystein-S-yl) acetaminophen, 2-methoxyacetaminophen sulfate, alliin, and daidzein sulfate, and generate a prediction of risk of cancer for the subject by applying a predictive model to the quantitative values of the plurality of biomarkers. In various embodiments, the metabolite biomarkers comprise three or more of pseudoephedrine, 3-(cystein-S-yl) acetaminophen, 2-methoxyacetaminophen sulfate, alliin, and daidzein sulfate. In various embodiments, the metabolite biomarkers comprise four or more of pseudoephedrine, 3-(cystein-S-yl) acetaminophen, 2-methoxyacetaminophen sulfate, alliin, and daidzein sulfate. In various embodiments, the metabolite biomarkers comprise each of pseudoephedrine, 3-(cystein-S-yl) acetaminophen, 2-methoxyacetaminophen sulfate, alliin, and daidzein sulfate. In various embodiments, the metabolite biomarkers further comprise one or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, the metabolite biomarkers further comprise five or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, the metabolite biomarkers further comprise ten or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, the metabolite biomarkers further comprise each of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

In various embodiments, the metabolite biomarkers further comprise one or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In various embodiments, the metabolite biomarkers further comprise five or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In various embodiments, the metabolite biomarkers further comprise ten or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In various embodiments, the metabolite biomarkers further comprise each of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3 alpha, 17beta-diol monosulfate, and homocitrulline.

In various embodiments, the cancer is lung cancer. In various embodiments, the risk of cancer is a level of risk of the subject developing cancer within 1 year, within 2 years, within 3 years, within 4 years, within 5 years, within 6 years, within 7 years, within 8 years, within 9 years, or within 10 years. In various embodiments, the risk of cancer is a presence or absence of cancer. In various embodiments, the level of risk is one of a low risk, medium risk, or high risk. In various embodiments, the dataset is derived from a test sample obtained from the subject. In various embodiments, the test sample is a blood or serum sample. In various embodiments, the test sample is obtained from having performed one or more assays. In various embodiments, the one or more assays comprise one or more of liquid chromatography (LC), gas chromatography (GC) (e.g., GC using an electron capture detector), a nitrogen/phosphorous detector, a flame photometric detector, high performance liquid chromatography (HPLC), nuclear magnetic resonance (NMR), mass spectrometry (MS), liquid chromatography MS (LC-MS), high performance LC-MS (HPLC-MS), or ultrahigh performance liquid chromatography-tandem MS (UPLC-MS/MS).

### BRIEF DESCRIPTION OF THE DRAWINGS

These and other features, aspects, and advantages of the present invention will become better understood with regard to the following description and accompanying drawings.

Figure (FIG. 1A depicts an overview of an environment for predicting risk of cancer in a subject via a cancer prediction system, in accordance with an embodiment.

FIG. 1B depicts a block diagram of the cancer prediction system, in accordance with an embodiment.

FIG. 2 depicts example training data for training a prediction model, in accordance with an embodiment.

FIG. 3 depicts implementation of an example prediction model, in accordance with a fourth embodiment.

FIG. 4 illustrates an example computer for implementing the entities shown in FIGS. 1A, 1B, 2, and 3.

FIG. 5 shows the performance of a binary predictor random forest predictive model as a function of the number of predictors in the model, in accordance with the embodiment of the prediction model shown in FIG. 3.

FIG. 6 shows the performance of a Cox Elastic net predictive model during training as a function of the number of predictors in the model, in accordance with the embodiment of the prediction model shown in FIG. 3.

### DETAILED DESCRIPTION

### I. Definitions

Terms used in the claims and specification are defined as set forth below unless otherwise specified.

The term “subject” encompasses a cell, tissue, or organism, human or non-human, whether in vivo, ex vivo, or in vitro, male or female.

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “predictor” or “predictors” refers to variables analyzed by a prediction model, or one or more panels of a prediction model. In various embodiments, a “predictor” refers to biomarkers, such as metabolite biomarkers.

The terms “marker,” “markers,” “biomarker,” and “biomarkers” encompass, without limitation, lipids, lipoproteins, proteins, cytokines, chemokines, growth factors, peptides, nucleic acids (e.g., DNA, mRNA, or micro-RNA (miRNA)), genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a prediction model, or are useful in prediction models developed using related markers (e.g., non-mutated versions of the proteins or nucleic acids, alternative transcripts, etc.). In particular embodiments, a marker or biomarker refers to a metabolite biomarker.

The term “antibody” is used in the broadest sense and specifically covers monoclonal antibodies (including full length monoclonal antibodies), polyclonal antibodies, multispecific antibodies (e.g., bispecific antibodies), and antibody fragments that are antigen-binding so long as they exhibit the desired biological activity, e.g., an antibody or an antigen-binding fragment thereof.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

A prediction model refers to a model that analyzes values for a plurality of predictors and determines a prediction of risk of cancer. In various embodiments, a prediction model includes one panel. In various embodiments, a prediction model includes more than one panel, such as two panels, three panels, four panels, five panels, six panels, seven panels, eight panels, nine panels, or ten panels. The two or more panels can provide combinable information for predicting risk of cancer for the subject.

The term “panel” refers to a set of predictors that are informative for predicting risk of cancer. In one example, quantitative values of biomarkers in a panel can be informative for predicting risk of cancer. In various embodiments, a panel can include two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five, forty six, forty seven, forty eight, forty nine, fifty, fifty one, fifty two, fifty three, fifty four, fifty five, fifty six, fifty seven, fifty eight, fifty nine, sixty, sixty one, sixty two, sixty three, sixty four, sixty five, sixty six, sixty seven, sixty eight, sixty nine, seventy, seventy one, seventy two, seventy three, seventy four, or seventy five predictors.

The term “obtaining a dataset associated with a sample” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

It must be noted that, as used in the specification, the singular forms “a,” “an” and “the” include plural referents unless the context clearly dictates otherwise.

### II. System Environment Overview

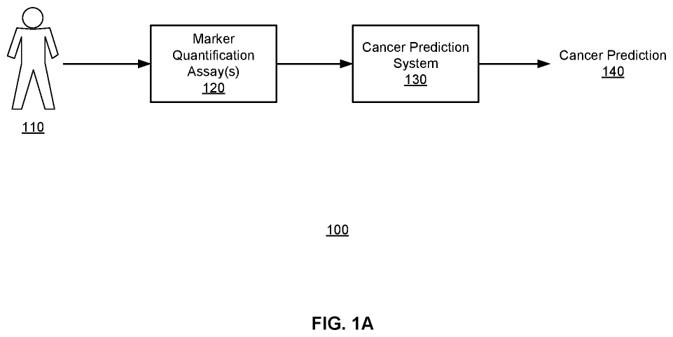

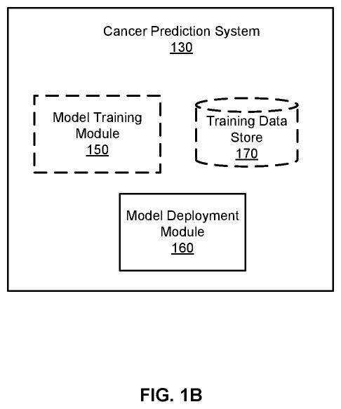

FIG. 1A depicts an overview of an environment 100 for predicting risk of cancer in a subject 110 via a cancer prediction system 130. The system environment 100 provides context in order to introduce a marker quantification assay 120 and a cancer prediction system 130 for determining a cancer prediction 140.

In various embodiments, a test sample is obtained from the subject 110. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other medical professional as would be known to one skilled in the art.

The test sample is tested to determine values of one or more biomarkers (e.g., metabolite biomarkers) by performing one or more marker quantification assays 120. A marker quantification assay 120 determines quantitative values of one or more biomarkers from the test sample. In various embodiments, more than one marker quantification assay 120 can be performed to determine values of one or more biomarkers. In particular embodiments, the marker quantification assay 120 is a metabolite quantification assay. Therefore, by performing the marker quantification assay 120, quantitative values of one or more metabolite biomarkers are determined.

In various embodiments, the marker quantification assay 120 may be an assay useful for detecting and/or quantifying metabolites in a biological sample. Example assays useful for detecting and/or quantifying metabolites in a biological sample include assays that employ liquid chromatography (LC), gas chromatography (GC) (e.g., GC using an electron capture detector), a nitrogen/phosphorous detector, a flame photometric detector, high performance liquid chromatography (HPLC), nuclear magnetic resonance (NMR), mass spectrometry (MS), or combinations thereof (e.g., liquid chromatography MS (LC-MS), high performance LC-MS (HPLC-MS), ultrahigh performance liquid chromatography-tandem MS (UPLC-MS/MS)). In various embodiments, the quantitative values of various biomarkers can be obtained in a single run using a single test sample obtained from the subject 110. In some embodiments, the quantitative values of biomarkers are obtained through multiple test samples obtained from the subject 110 (e.g., a blood sample). The quantified values of the biomarkers are provided to the cancer prediction system 130.

Generally, the cancer prediction system 130 analyzes the quantitative values of biomarkers (e.g., metabolite biomarkers) determined by the marker quantification assay(s) 120 and generates the cancer prediction 140. In various embodiments, the cancer prediction 140 represents a prediction of presence or absence of cancer in the subject. In various embodiments, the cancer prediction 140 can be a future risk of cancer prediction for the subject 110 (e.g., a likelihood of the subject developing cancer within a time period e.g., within 1-5 years). In various embodiments, the cancer prediction 140 can be a risk of cancer prediction for the subject 110 (e.g., a presence or absence of cancer in the subject 110). In various embodiments, the cancer prediction 140 can be informative for identifying a therapeutic that is likely to be effective in treating a cancer that is present or is predicted to occur within a predetermined time. In various embodiments, the therapeutic can serve as a prophylactic to delay or prevent the onset of the cancer within the predetermined time.

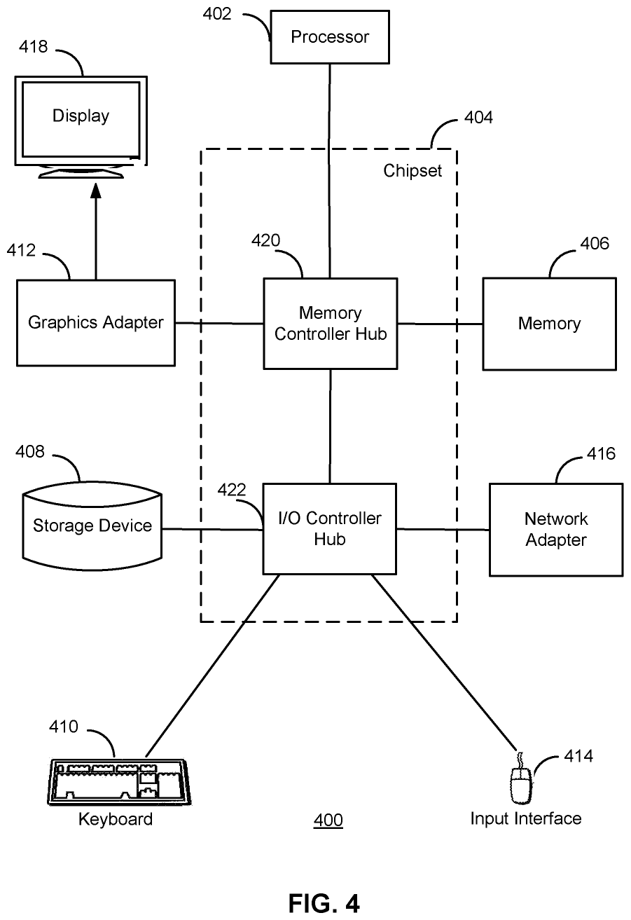

The cancer prediction system 130 can include one or more computers, embodied as a computer system 400 as discussed below with respect to FIG. 4. Therefore, in various embodiments, the steps described in reference to the cancer prediction system 130 are performed in silico.

In various embodiments, the marker quantification assay 120 and the cancer prediction system 130 can be employed by different parties. For example, a first party performs the marker quantification assay 120 and then provides the determined quantitative values to a second party which implements the cancer prediction system 130. For example, the first party may be a clinical laboratory that obtains test samples from subjects 110 and performs marker quantification assay(s) 120 on the test samples. The second party receives the quantitative values of biomarkers resulting from performed marker quantification assay(s) 120 and analyzes the quantitative values using the cancer prediction system 130.

Reference is now made to FIG. 1B which depicts a block diagram illustrating the computer logic components of the cancer prediction system 130, in accordance with an embodiment. Specifically, the cancer prediction system 130 may include a model training module 150, a model deployment module 160, and a training data store 170.

Each of the components of the cancer prediction system 130 is hereafter described in reference to two phases: 1) a training phase and 2) a deployment phase. More specifically, the training phase refers to the building and training of one or more prediction models based on training data that includes quantitative values of biomarkers obtained from individuals that are known to be healthy (e.g., absence of cancer), known to have cancer (e.g., previously diagnosed with cancer), or known to develop cancer within a certain amount of time (e.g., within 1-5 years). Therefore, the prediction models are trained to predict a risk of cancer in a subject based on at least quantitative biomarker values.

During the deployment phase, a prediction model is applied to quantitative biomarker values (e.g., metabolite biomarker values) from a test sample obtained from a subject of interest to predict risk of cancer for the subject of interest. In various embodiments, the prediction model only analyzes quantitative biomarker values from a test sample obtained from the subject.

In some embodiments, the components of the cancer prediction system 130 are applied during one of the training phase and the deployment phase. For example, the model training module 150 and training data store 170 (indicated by the dotted lines in FIG. 1B) are applied during the training phase whereas the model deployment module 160 is applied during the deployment phase. In various embodiments, the components of the cancer prediction system 130 can be performed by different parties depending on whether the components are applied during the training phase or the deployment phase. In such scenarios, the training and deployment of the prediction model are performed by different parties. For example, the model training module 150 and training data store 170 applied during the training phase can be employed by a first party (e.g., to train a prediction model) and the model deployment module 160 applied during the deployment phase can be performed by a second party (e.g., to deploy the prediction model).

### III. Prediction Model

### III.A. Training a Prediction Model

During the training phase, the model training module 150 trains one or more prediction models using training data. In various embodiments, the training data can be derived from samples obtained from individuals. In various embodiments, the training data includes quantitative values of biomarkers (e.g., metabolite biomarkers) derived from the samples obtained from individuals. Such individuals can be healthy individuals, individuals known to have cancer (e.g., individuals previously diagnosed with cancer), or individuals that are known to develop cancer within a particular timeframe. In various embodiments, the individuals from which training data are derived are clinical subjects. For example, the training data can include quantitative values of biomarkers (e.g., metabolite biomarkers) that were measured from test samples obtained from clinical subjects, such as subjects that were enrolled in a clinical study or clinical trial.

Referring to FIG. 1B, the training data may be stored in the training data store 170. In various embodiments, the cancer prediction system 130 generates the training data and analyzes quantitative values of biomarkers from test samples. In various embodiments, the cancer prediction system 130 obtains the training data from a third party. The third party may have analyzed test samples to determine the quantitative biomarker values from the individuals.

In various embodiments, the training data includes reference ground truths that indicate information about a cancer. As an example, the training data can include a reference ground truth that indicates a presence or absence of cancer. As another example, the training data can include a reference ground truth that indicates development of cancer within a certain time. For example, the training data can include a reference ground truth that indicates that a subject developed cancer within a particular time period. In various embodiments, the time period can be any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years. In various embodiments, the training data can include two or more reference ground truths, each reference ground truth indicating development of cancer within a particular timeframe. For example, the training data can include a first reference ground truth indicating whether the individual developed cancer within 1 year and can further include a second reference ground truth indicating whether the individual developed cancer within 3 years.

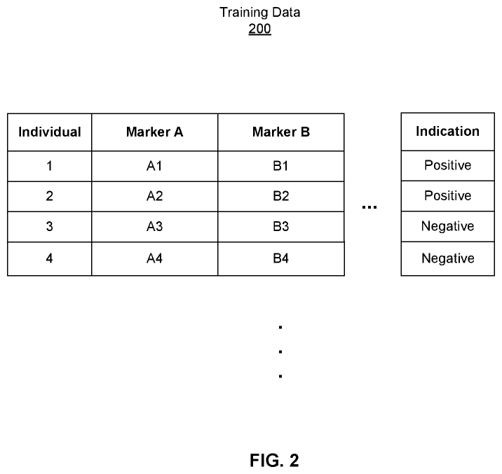

Reference is made to FIG. 2, which depicts an example set of training data 200, in accordance with an embodiment. As shown in FIG. 2, the training data 200 includes data corresponding to multiple individuals (e.g., column 1 depicting individual 1, 2, 3, 4 . . . ). For each individual, the training data 200 includes quantitative values (e.g., A1, B1, A2, B2, etc.) for different markers (e.g., metabolite biomarkers) obtained from the corresponding individual. In some embodiments, the quantitative values are determined by the marker quantification assay 120 shown in FIG. 1A. Although FIG. 2 explicitly depicts four individuals and two different markers (marker A and marker B), the training data 200 may include tens, hundreds, or thousands of individuals, tens, hundreds, or thousands of markers.

As shown in FIG. 2, a first training example (e.g., first row) of the training data refers to individual 1, corresponding quantitative values of marker A (e.g., A1) and marker B (e.g., B1). Similarly, the second training example (e.g., second row) of the training data refers to individual 2, corresponding quantitative values of marker A (e.g., A2) and marker B (e.g., B2). Individuals 3 and 4 have similar corresponding marker values as shown in FIG. 2.

The training data 200 further includes a reference ground truth (e.g., column titled “Indication”) that indicates cancer information pertaining to the corresponding individual. As an example, an indication may be a current presence or current absence of cancer in the individual. As another example, an indication may be a presence or absence of cancer in the individual within a time period. For example, referring to the first training example (e.g., first row), a “Positive” indication under the column titled “Time” can indicate that the individual 1 developed cancer within the time period (e.g., within any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years). Referring to the second training example (e.g., second row), the second training example includes an indication of “Positive” under the column titled “Indication” which indicates that the second individual developed cancer within the time period. The third and fourth training examples corresponding to Individual 3 and Individual 4, respectively, include reference ground truths with an indication of “Negative” which indicates that the individuals do not develop cancer within the time period.

Although the training data 200 in FIG. 2 depicts one reference ground truth (e.g., “Indication”), in various embodiments, training data 200 can include more reference ground truths (e.g., two indications or more). As one example, the training data 200 can additionally include reference ground truth values that indicate whether the individual developed cancer within two, three, four, five, six, seven, eight, nine, ten, eleven, twelve, thirteen, fourteen, fifteen, sixteen, seventeen, eighteen, nineteen, or twenty other time periods.

In some embodiments, for training the prediction model, the model training module 150 retrieves the training data from the training data store 170 and randomly partitions the training data into a training set and a test set. As an example, 66% of the training data may be partitioned into the training set and the other 33% can be partitioned into the test set. Other proportions of training set and test set may be implemented. As such, the training set is used to train prediction models whereas the test set is used to validate the prediction models.

In various embodiments, the prediction model is any one of a regression model (e.g., linear regression, logistic regression, Cox regression, elastic net regression, Cox Elastic regression model, ridge regression, or polynomial regression), decision tree, random forest, support vector machine, elastic net regulation, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof.

The prediction model can be trained using a machine learning implemented method, such as any one of a linear regression algorithm, logistic regression algorithm, decision tree algorithm, support vector machine classification, elastic net regulation, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the prediction model is trained using supervised learning algorithms, unsupervised learning algorithms, semi-supervised learning algorithms (e.g., partial supervision), weak supervision, transfer, multi-task learning, or any combination thereof.

In various embodiments, the prediction model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the prediction model are trained (e.g., adjusted) using the training data to improve the predictive capacity of the prediction model.

The model training module 150 trains a prediction model using the training data. In various embodiments, the model training module 150 constructs a prediction model that receives, as input, two or more predictors (e.g., values of biomarkers). In various embodiments, the model training module 150 constructs a prediction model that receives, as input, three predictors. In various embodiments, the model training module 150 constructs a prediction model that receives, as input, four predictors. In various embodiments, the model training module 150 constructs a prediction model that receives, as input, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five, forty six, forty seven, forty eight, forty nine, fifty, fifty one, fifty two, fifty three, fifty four, fifty five, fifty six, fifty seven, fifty eight, fifty nine, sixty, sixty one, sixty two, sixty three, sixty four, sixty five, sixty six, sixty seven, sixty eight, sixty nine, seventy, seventy one, seventy two, seventy three, seventy four, or seventy five or more predictors.

In various embodiments, the model training module 150 constructs a prediction model that receives, as input, quantitative values of three biomarkers. In various embodiments, the model training module 150 constructs a prediction model that receives, as input, quantitative values of four biomarkers. In some embodiments, the model training module 150 constructs a prediction model that receives, as input, quantitative values for more than four biomarkers. In various embodiments, the model training module 150 constructs a prediction model that receives as input, quantitative values for five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five, forty six, forty seven, forty eight, forty nine, or fifty or more markers. In particular embodiments, the model training module 150 constructs a prediction model that receives as input, quantitative values for 5 markers. In particular embodiments, the model training module 150 constructs a prediction model that receives as input, quantitative values for at least 10 markers. In particular embodiments, the model training module 150 constructs a prediction model that receives as input, quantitative values for at least 20 markers. In particular embodiments, the model training module 150 constructs a prediction model that receives as input, quantitative values for at least 30 markers. In particular embodiments, the model training module 150 constructs a prediction model that receives as input, quantitative values for at least 40 markers. In particular embodiments, the model training module 150 constructs a prediction model that receives as input, quantitative values for at least any of 5, 10, 20, 30, or 34 biomarkers.

In various embodiments, the model training module 150 identifies a set of biomarkers that are to be used to train a prediction model. The model training module 150 may begin with a list of candidate biomarkers that are promising for diagnosing a cancer. In various embodiment, the model training module 150 performs a feature selection process to identify the set of biomarkers to be included for the prediction model. For example, candidate biomarkers that are determined to be highly correlated with a presence of cancer would be deemed important are therefore likely to be included in the panel in comparison to other biomarkers that are not highly correlated.

In various embodiments, each prediction model is iteratively trained using, as input, the quantitative values of the markers for each individual. For example, referring again to FIG. 2, one iteration involves providing a training example (e.g., a row of the training data). Each prediction model is trained on reference ground truth data that includes the indication(s). In various embodiments, over training iterations, the prediction model is trained (e.g., the parameters are tuned) to minimize a prediction error between a prediction outputted by the prediction model and the ground truth data. In various embodiments, the prediction error is calculated based on a loss function, examples of which include a L1 regularization (Lasso Regression) loss function, a L2 regularization (Ridge Regression) loss function, or a combination of L1 and L2 regularization (ElasticNet).

In various embodiments, a penalty factor is employed to lower the risk of false-positive selection of predictive biomarkers arising from their low levels. In various embodiments, a penalty factor is added to the general Elastic Net penalty based on the proportion of values of each biomarker at or below a lower limit of quantitation (LLOQ).

### III.B. Deploying a Prediction Model

During the deployment phase, the model deployment module 160 (as shown in FIG. 1B) applies a trained prediction model to generate a prediction for risk of cancer in the subject. In various embodiments, the prediction for risk of cancer for the subject is a prediction of presence of absence of cancer in the subject. In particular embodiments, the subject has not previously been diagnosed with a disease. Therefore, the deployment of the prediction model enables in silico prediction of whether the subject is likely to develop cancer in the future (e.g., within 1-20 years). In various embodiments, the model deployment module 160 applies a trained prediction model that analyzes quantitative values of biomarkers to determine a risk of cancer in a subject.

In various embodiments, the trained prediction model includes a single panel that includes one or more biomarkers. Thus, the trained prediction model outputs a prediction based on the one or more biomarkers of the single panel.

In various embodiments, the trained prediction model includes two or more panels, each panel comprising one or more biomarkers. In various embodiments, a panel includes a set of biomarkers that are distinct from a set of biomarkers of another panel in the prediction model. In various embodiments, one or more biomarkers of one panel can overlap with one or more biomarkers of another panel. In other words, two panels may share one or more biomarkers. In various embodiments, two panels may share one, two three, four, five, six, seven, eight, nine, or ten biomarkers. In particular embodiments, two panels share five biomarkers.

In such embodiments where the trained prediction model includes two or more panels, the trained prediction model outputs a prediction based on the biomarkers of each of the two or more panels. To generate an overall prediction, the trained prediction model combines an output of a first panel with an output of a second panel. Thus, the one or more biomarkers of the first panel as well as the one or more biomarkers of the second panel contribute towards the overall prediction outputted by the trained prediction model.

In various embodiments, the output of each of the panels of the prediction model is a score (e.g. an indication of how likely it is that the subject has cancer or will develop cancer). Thus, the trained prediction model combines scores outputted by the individual panels to generate an overall prediction. In various embodiments, the trained prediction model combines the scores outputted by the individual panels by comparing the scores outputted by the individual panels and selecting one of the scores. Thus, the selected score serves as the basis for the overall prediction of the prediction model. In various embodiments, the trained prediction model combines the scores outputted by the individual panels by comparing the scores outputted by the individual panels and selecting the higher score.

In various embodiments, the trained prediction model combines the supplemented scores by comparing the supplemented scores and selecting one of the supplemented scores. In various embodiments, the prediction model selects the highest supplemented score. In such embodiments, the overall prediction outputted by the prediction model can be the selected score or can be derived from the selected score (e.g., overall prediction is generated based on the comparison between the selected score and a reference score as described above).

In various embodiments, prior to comparing the scores and selecting a score, the prediction model normalizes each score outputted by a panel to a corresponding reference score. Thus, normalized scores are compared to one another to select the score.

In various embodiments, the overall prediction outputted by the prediction model is the selected score that is selected from the scores outputted the panels. In various embodiments, the prediction model generates the overall prediction by comparing the selected score to one or more reference scores. In various embodiments, the reference score can be a score corresponding to healthy patients (e.g., a “healthy score”), a baseline score at a prior timepoint (e.g., longitudinal analysis), a score corresponding to patients clinically diagnosed with cancer (e.g., a “reference cancer score”), a score corresponding to patients diagnosed with a particular subtype of cancer (e.g., a cancer subtype score), a score corresponding to patients who are known to develop cancer within a particular time period (e.g., a time to event score), or a threshold score (e.g., a cutoff).

In particular embodiments, the reference score can be a “healthy score” corresponding to healthy patients and can be generated by implementing a prediction model to analyze quantitative values of biomarkers. In particular embodiments, the reference score is a time to event score corresponding to patients who are known to develop cancer within a time period (e.g., within any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years).

In various embodiments, the overall prediction is generated based on the comparison between a score of the prediction model and one or more reference scores. The overall prediction is informative for predicting risk of cancer for the subject within one or more time periods. To provide an example, the score can be from a panel of the prediction model. The score is compared to a healthy score (e.g., reference score derived from healthy patients). If the score is significantly different (e.g., p<0.05) from the healthy score, the overall prediction can indicate that the subject has cancer, or will likely develop cancer. As another example, the score from the prediction model can be compared to one or more time to event scores of patients who are known to develop cancer within a particular time period. If the score is significantly different (e.g., p<0.05) from a time to event score, then the overall prediction can indicate that the subject is unlikely to develop cancer within a period of time corresponding to the time to event score. If the score is not significantly different (e.g., p>0.05) from a time to event score, then the overall prediction can indicate that the subject is likely to develop cancer within a period of time corresponding to the time to event score. As described herein, a period of time can be any of within any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years.

In various embodiments, the subject can undergo treatment depending on the overall prediction. For example, if the subject is predicted to likely develop cancer within a particular period of time, the subject can be administered a therapeutic intervention. Here, the therapeutic intervention can serve as a prophylactic treatment to delay or prevent the onset of the cancer.

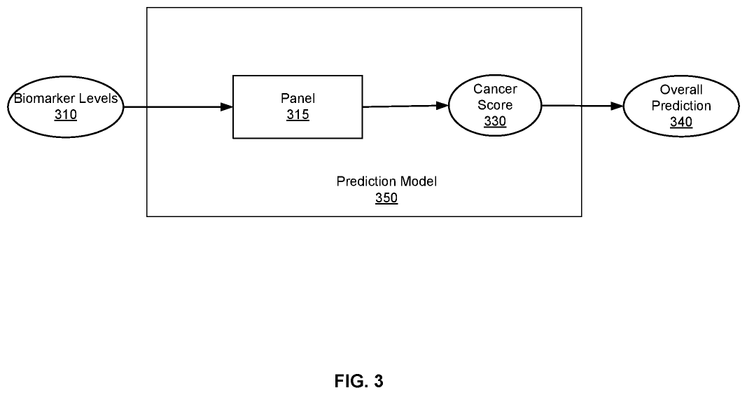

Reference is now made to FIG. 3, which depicts implementation of an example prediction model, in accordance with a fourth embodiment. Here, the prediction model 350 may include a single panel 315. Thus, single panel 315 of the prediction model analyzes the quantitative biomarker levels 310.

Based on the analysis of the quantitative biomarker levels 310, the prediction model 350 generates a cancer score 330. The cancer score 330 is compared to one or more reference scores. In various embodiments, the cancer score 330 can be compared to a time to event score. If the cancer score 330 is not significantly different (e.g., p>0.05) from the time to event score, then the overall prediction 340 can indicate that the individual is likely to develop cancer within a time period corresponding to the time to event score. Alternatively, if the cancer score 330 is significantly different (e.g., p<0.05) from the time to event score, then the overall prediction 340 can indicate that individual is not likely to develop cancer within the time period corresponding to the time to event score. The cancer score 330 can be compared to multiple time to event scores corresponding to different time periods to predict whether the individual is likely to develop cancer within any of the time periods corresponding to the time to event scores.

As shown and described in reference to FIG. 3, the prediction model 350 can generate a cancer score (e.g., cancer score 330) that is informative for determining an overall prediction 340. In various embodiments, the cancer score represents an aggregate score of the levels (e.g., altered or dysregulated levels) of the biomarkers of the prediction model 350. This means that it is not necessary to know how the level of any individual marker has changed to obtain the cancer score. For example, assuming a prediction model of 20 biomarkers, the upregulation or downregulation of any one biomarker represents one component that results in the cancer score. Thus, even though a first patient and second patient may both exhibit upregulation of a biomarker, the final aggregate cancer scores may indicate that the first patient is likely to develop cancer within a certain timeframe, whereas the second patient is unlikely to develop cancer within the certain timeframe.

As further shown in FIG. 3, the output of the prediction model 350 is an overall prediction 340. In particular embodiments, the overall prediction 340 represents a prediction of risk of cancer (e.g., lung cancer) for the subject. In particular embodiments, the overall prediction 340 represents a prediction of whether the subject is likely to develop lung cancer within a particular time period. In various embodiments, the time period is any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years. In various embodiments, the overall prediction 340 can represent multiple predictions of whether the subject is likely to develop lung cancer within N different time periods. In various embodiments, N is 1, 2, 3, 4, 5, 6, 7, 8, 9, 10, 11, 12, 13, 14, 15, 16, 17, 18, 19, or 20 different time periods.

### IV. Panel(s) of a Prediction Model

Embodiments described herein involve implementing a prediction model that includes one or more panels. Each panel includes one or more predictors, examples of which include biomarkers (e.g., metabolite biomarkers).

In various embodiments, multiple panels can be included in a prediction model. The implementation of multiple panels is informative for generating an overall prediction for risk of cancer in a subject. In various embodiments, a panel of the prediction model is a univariate panel. In such embodiments, the univariate panel includes one predictor. In other embodiments, a panel is a multivariate panel. In such embodiments, the multivariate panel includes more than one predictor. In various embodiments, the multivariate panel includes two predictors. In various embodiments, the multivariate panel includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 or more predictors. In particular embodiments, the multivariate panel includes five predictors. In particular embodiments, the multivariate panel includes ten predictors. In particular embodiments, the multivariate panel includes twenty predictors. In particular embodiments, the multivariate panel includes thirty predictors. In particular embodiments, the multivariate panel includes thirty four predictors.

Referring to FIG. 3, in various embodiments, panel 315 includes between 1 and 25 biomarkers. In various embodiments, panel 315 includes between 2 and 15 biomarkers. In various embodiments, panel 315 includes between 3 and 12 biomarkers. In various embodiments, panel 315 includes between 4 and 10 biomarkers. In particular embodiments, panel 315 includes 8 biomarkers. In various embodiments, panel 315 includes between 1 and 25 biomarkers. In various embodiments, panel 315 includes between 5 and 21 biomarkers. In various embodiments, panel 315 includes between 10 and 20 biomarkers. In various embodiments, panel 315 includes between 14 and 19 biomarkers. In particular embodiments, panel 315 includes 15 biomarkers. In particular embodiments, panel 315 includes 17 biomarkers.

In various embodiments, the prediction model (such as the prediction model in FIG. 3) includes between 1 and 60 biomarkers. In various embodiments, the prediction model includes between 10 and 50 biomarkers. In various embodiments, the prediction model includes between 20 and 40 biomarkers. In various embodiments, the prediction model includes between 25 and 38 biomarkers. In various embodiments, the prediction model includes between 30 and 35 biomarkers. In various embodiments, the prediction model includes between 20 and 30 biomarkers. In various embodiments, the prediction model includes between 30 and 40 biomarkers. In various embodiments, the prediction model includes between 40 and 50 biomarkers. In particular embodiments, the prediction model includes 5 biomarkers. In particular embodiments, the prediction model includes 10 biomarkers. In particular embodiments, the prediction model includes 20 biomarkers. In particular embodiments, the prediction model includes 34 biomarkers. In particular embodiments, the prediction model includes 36 biomarkers.

In various embodiments, a panel of the prediction model (such as the panel of the prediction model shown in any of FIG. 3) includes one or more metabolite biomarkers. Example metabolite biomarkers included in panels of the prediction model or the prediction model include metabolite biomarkers shown below in Table 1 or Table 2.

In various embodiments, a panel of the prediction model (such as the panel of the prediction model shown in any of FIG. 3) includes two or more metabolite biomarkers selected from beta-hydroxyisovaleroylcarnitine, pyrraline, citramalate, succinate, urate, 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, threonine, 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), salicyluric glucuronide. In various embodiments, panels of the prediction model (such as panels of the prediction model shown in FIG. 3) include two or more metabolite biomarkers selected from pseudoephedrine, 3-(cystein-S-yl) acetaminophen, 2-methoxyacetaminophen sulfate, alliin, daidzein sulfate, alpha-ketoglutarate, sedoheptulose, 1-cerotoyl-GPC (26:0), 3-hydroxy-2-methylpyridine sulfate, cysteine sulfinic acid, docosahexaenoylcholine, stearoylcholine, glucuronide of C10H18O2, N-carbamoylalanine, cyclo (phe-pro), 4-acetamidophenol, allantoin, salicyluric glucuronide, pyrraline, 3-hydroxycotinine glucuronide, 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3 alpha, 17beta-diol monosulfate, and homocitrulline.

In various embodiments, a panel of the prediction model (such as the panel of the prediction model shown in any of FIG. 3) includes three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, thirty or more, thirty one or more, thirty two or more, thirty three or more, thirty four or more, or thirty five or more metabolite biomarkers selected from beta-hydroxyisovaleroylcarnitine, pyrraline, citramalate, succinate, urate, 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, threonine, 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), salicyluric glucuronide. In various embodiments, panels of the prediction model (such as panels of the prediction model shown in FIG. 3) include three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, thirty or more, thirty one or more, thirty two or more, thirty three or more, thirty four or more, or thirty five or more metabolite biomarkers selected from pseudoephedrine, 3-(cystein-S-yl) acetaminophen, 2-methoxyacetaminophen sulfate, alliin, daidzein sulfate, alpha-ketoglutarate, sedoheptulose, 1-cerotoyl-GPC (26:0), 3-hydroxy-2-methylpyridine sulfate, cysteine sulfinic acid, docosahexaenoylcholine, stearoylcholine, glucuronide of C10H18O2, N-carbamoylalanine, cyclo (phe-pro), 4-acetamidophenol, allantoin, salicyluric glucuronide, pyrraline, 3-hydroxycotinine glucuronide, 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

In particular embodiments, a panel of the prediction model (such as the panel of the prediction model shown in any of FIG. 3) includes each of beta-hydroxyisovaleroylcarnitine, pyrraline, citramalate, succinate, urate, 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, threonine, 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), salicyluric glucuronide. In particular embodiments, panels of the prediction model (such as panels of the prediction model shown in FIG. 3) include each of pseudoephedrine, 3-(cystein-S-yl) acetaminophen, 2-methoxyacetaminophen sulfate, alliin, daidzein sulfate, alpha-ketoglutarate, sedoheptulose, 1-cerotoyl-GPC (26:0), 3-hydroxy-2-methylpyridine sulfate, cysteine sulfinic acid, docosahexaenoylcholine, stearoylcholine, glucuronide of C10H18O2, N-carbamoylalanine, cyclo (phe-pro), 4-acetamidophenol, allantoin, salicyluric glucuronide, pyrraline, 3-hydroxycotinine glucuronide, 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

In particular embodiments, a panel of the prediction model (such as the panel of the prediction model shown in any of FIG. 3) includes three or more of Beta-hydroxyisovaleroylcarnitine, Pyrraline, Citramalate, Succinate, and Urate. In particular embodiments, panels of the prediction model include four or more of Beta-hydroxyisovaleroylcarnitine, Pyrraline, Citramalate, Succinate, and Urate. In particular embodiments, panels of the prediction model (such as panels of the prediction model shown in FIG. 3) include each of beta-hydroxyisovaleroylcarnitine, pyrraline, citramalate, succinate, and urate. In particular embodiments, panels of the prediction model (such as panels of the prediction model shown in FIG. 3) include each of beta-hydroxyisovaleroylcarnitine, pyrraline, citramalate, succinate, urate, 2-aminophenol sulfate, guanidinosuccinate, docosahexaenoylcholine, sphingomyelin (d18:2/18:1), and homocitrulline.

In particular embodiments, a panel of the prediction model (such as the panel of the prediction model shown in any of FIG. 3) includes one or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In particular embodiments, panels of the prediction model (such as panels of the prediction model shown in FIG. 3) include five or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In particular embodiments, panels of the prediction model (such as panels of the prediction model shown in FIG. 3) include ten or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine. In particular embodiments, panels of the prediction model (such as panels of the prediction model shown in FIG. 3) include each of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

In various embodiments, a panel of the prediction model (such as the panel of the prediction model shown in any of FIG. 3) includes one or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, panels of the prediction model (such as panels of the prediction model shown in FIG. 3) include five or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, panels of the prediction model (such as panels of the prediction model shown in FIG. 3) include ten or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide. In various embodiments, panels of the prediction model (such as panels of the prediction model shown in FIG. 3) include each of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

In various embodiments, a panel of the prediction model (such as the panel of the prediction model shown in any of FIG. 3) includes two or more of pseudoephedrine, 3-(cystein-S-yl) acetaminophen, 2-methoxyacetaminophen sulfate, alliin, and daidzein sulfate. In particular embodiments, panels of the prediction model include three or more of pseudoephedrine, 3-(cystein-S-yl) acetaminophen, 2-methoxyacetaminophen sulfate, alliin, and daidzein sulfate. In particular embodiments, panels of the prediction model (such as panels of the prediction model shown in FIG. 3) include four or more of pseudoephedrine, 3-(cystein-S-yl) acetaminophen, 2-methoxyacetaminophen sulfate, alliin, and daidzein sulfate. In particular embodiments, panels of the prediction model (such as panels of the prediction model shown in FIG. 3) include each of pseudoephedrine, 3-(cystein-S-yl) acetaminophen, 2-methoxyacetaminophen sulfate, alliin, and daidzein sulfate.

In particular embodiments, a panel of the prediction model (such as the panel of the prediction model shown in any of FIG. 3) includes each of pseudoephedrine, 3-(cystein-S-yl) acetaminophen, 2-methoxyacetaminophen sulfate, alliin, daidzein sulfate, alpha-ketoglutarate, sedoheptulose, 1-cerotoyl-GPC (26:0), 3-hydroxy-2-methylpyridine sulfate, and cysteine sulfinic acid. In various embodiments, panels of the prediction model include one or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, panels of the prediction model include five or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, panels of the prediction model include ten or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide. In various embodiments, panels of the prediction model include each of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H1802, N-carbamoylalanine, cyclo (phe-pro), 4-acetamidophenol, allantoin, salicyluric glucuronide, pyrraline, and 3-hydroxycotinine glucuronide. In particular embodiments, panels of the prediction model (such as panels of the prediction model shown in FIG. 3) include each of pseudoephedrine, 3-(cystein-S-yl) acetaminophen, 2-methoxyacetaminophen sulfate, alliin, daidzein sulfate, alpha-ketoglutarate, sedoheptulose, 1-cerotoyl-GPC (26:0), 3-hydroxy-2-methylpyridine sulfate, cysteine sulfinic acid, docosahexaenoylcholine, stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

In particular embodiments, a panel of the prediction model (such as the panel of the prediction model shown in any of FIG. 3) includes one or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In particular embodiments, panels of the prediction model (such as panels of the prediction model shown in FIG. 3) include five or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In particular embodiments, panels of the prediction model (such as panels of the prediction model shown in FIG. 3) include ten or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline. In particular embodiments, panels of the prediction model (such as panels of the prediction model shown in FIG. 3) include each of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

### V. Assays

As shown in FIG. 1A, the system environment 100 involves implementing a marker quantification assay 120 for evaluating quantitative values of one or more biomarkers. Examples of an assay (e.g., marker quantification assay 120) for one or more markers include assays that employ liquid chromatography (LC), gas chromatography (GC) (e.g., GC using an electron capture detector), a nitrogen/phosphorous detector, a flame photometric detector, high performance liquid chromatography (HPLC), nuclear magnetic resonance (NMR), mass spectrometry (MS), or combinations thereof (e.g., liquid chromatography MS (LC-MS), high performance LC-MS (HPLC-MS), ultrahigh performance liquid chromatography-tandem MS (UPLC-MS/MS)).

The information from the assay can be quantitative and sent to a computer system of the invention. The information can also be qualitative, such as observing patterns or fluorescence, which can be translated into a quantitative measure by a user or automatically by a reader or computer system.

In various embodiments, prior to implementation of a marker quantification assay 120, a sample obtained from a subject can be processed. In various embodiments, processing the sample enables the implementation of the marker quantification assay 120 to more accurately evaluate quantitative values of one or more biomarkers in the sample.

In various embodiments, the sample from a subject can be processed to extract biomarkers from the sample. In one embodiment, the sample can undergo phase separation to separate the biomarkers from other portions of the sample. For example, the sample can undergo centrifugation (e.g., pelleting or density gradient centrifugation) to separate larger and/or more dense entities in the sample (e.g., cells and other macromolecules) from the biomarkers. Other examples include filtration (e.g., ultrafiltration) to phase separate the biomarkers from other portions of the sample.

In various embodiments, the sample from a subject can be processed to produce a sub-sample with a fraction of biomarkers that were in the sample. In various embodiments, producing a fraction of biomarkers can involve performing a fractionation procedure. One example of fractionation procedures include chromatography (e.g., gel filtration, ion exchange, hydrophobic chromatography, liquid chromatography or affinity chromatography). In particular embodiments, the protein fractionation procedure involves affinity purification or immunoprecipitation where biomarkers are bound by specific antibodies. Such antibodies can be immobilized on a support, such as a magnetic particle or nanoparticle or a plate.

### VI. Therapeutic Agents and Compositions for Therapeutic Agents

In various embodiments, a therapeutic agent can be provided to a subject subsequent to obtaining the sample from the subject and determining quantitative values of one or more markers in the obtained sample. As one example, a prediction model that analyzes predictors including quantitative values of one or more markers predicts that an individual is likely to develop cancer within a time period. In various embodiments, the prediction model may generate a prediction that is informative for selecting a therapeutic agent to be provided to the subject, the therapeutic agent likely to delay or prevent the onset of the cancer within the time period. For example, if the prediction model predicts that the subject has a presence of cancer, the prediction from the prediction model can be used to select a therapeutic agent for treating the currently present cancer. As another example, if the prediction model predicts that the subject is likely to develop cancer within a future timeframe, the prediction from the prediction model can be used to select a therapeutic agent that can be administered prophylactically (e.g., to prevent or to slow the onset of the future development of the cancer).

In various embodiments the therapeutic agent is a biologic, e.g. a cytokine, antibody, soluble cytokine receptor, anti-sense oligonucleotide, siRNA, RNA/DNA based vaccine, immune cell based therapies (e.g., adoptive cell therapy), and the like. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists. Also included are biosimilar or bioequivalent drugs to the active agents set forth herein. In various embodiments, the therapeutic agent can be radiotherapy or a surgical intervention.

Therapeutic agents for lung cancer can include chemotherapeutics such as docetaxel, doxorubicin hydrocholoride, methotrexate, cisplatin, carboplatin, gemcitabine, Nab-paclitaxel, paclitaxel, pemetrexed, gefitinib, erlotinib, brigatinib (Alunbrig®), capmatinib (Tabrecta®), selpercatinib (Retevmo®), entrectinib (Rozlytrek®), lorlatinib (Lorbrena®), larotrectinib (Vitrakvi®), dacomitinib (Vizimpro®), everolimus (Afinitor®), vinorelbine, pralsetinib (Gavreto®), dabrafenib (Tafinlar®), trametinib (Mekinist®), crizotinib (Xalkori®), alectinib (Alecensa®), ceritinib (Zykadia®), osimertinib (Tagrisso®). Afatinib (Gilotrif®), dacomitinib (Vizimpro®), and nintedanib (Vargatef®). Therapeutic agents for lung cancer can include antibody therapies such as durvalumab (Imfinzi®), nivolumab (Opdivo®), pembrolizumab (Keytruda®), atezolizumab (Tecentriq®), ramucirumab, bevacizumab (Avastin®, Mvasi®, Zirabev®), necitumumab (Portrazza®), and ipilimumab (Yervoy®).

A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients. Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

Such a pharmaceutical composition may be administered for treatment (e.g., after diagnosis of a patient with lung cancer) purposes. Preventing, prophylaxis or prevention of a disease or disorder as used in the context of this invention refers to the administration of a composition to prevent the occurrence, onset, progression, or recurrence of lung cancer some or all of the symptoms of lung cancer or to lessen the likelihood of the onset of lung cancer. Treating, treatment, or therapy of lung cancer shall mean slowing, stopping or reversing the cancer's progression by administration of treatment according to the present invention. In the preferred embodiment, treating lung cancer means reversing the cancer's progression, ideally to the point of eliminating the cancer itself.

### VII. Cancers

Methods described herein involve diagnosing a cancer in a subject. In various embodiments, the cancer in the subject can include one or more of: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancer, testicular cancer, colon and/or rectal cancer, prostatic cancer, or pancreatic cancer.

### VIII. Computer Implementation

The methods of the invention, including the methods of predicting risk of cancer in an individual, are, in some embodiments, performed on one or more computers.

For example, the building and deployment of a prediction model and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of a prediction model. Such data can be used for a variety of purposes, such as patient monitoring, treatment considerations, and the like. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods of predicting risk of cancer in an individual, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### VIII.A. Example Computer

FIG. 4 illustrates an example computer for implementing the entities shown in FIGS. 1A, 1B, 2, and 3. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard 410, or some combination thereof, and is used to input data into the computer 400. In some embodiments, the computer 400 may be configured to receive input (e.g., commands) from the input interface 414 via gestures from the user. The graphics adapter 412 displays images and other information on the display 418. The network adapter 416 couples the computer 400 to one or more computer networks.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIGS. 1A, 1B, and 2 can vary depending upon the embodiment and the processing power required by the entity. For example, the cancer prediction system 130 can run in a single computer 400 or multiple computers 400 communicating with each other through a network such as in a server farm. The computers 400 can lack some of the components described above, such as graphics adapters 412, and displays 418.

### IX. Kit Implementation

Also disclosed herein are kits for predicting risk of a cancer in an individual. Such kits can include reagents for detecting quantitative values of one or biomarkers and instructions for predicting risk of cancer based on at least the detected quantitative values of the biomarkers.

The detection reagents can be provided as part of a kit. Thus, the invention further provides kits for detecting the presence of a panel of biomarkers of interest in a biological test sample. A kit can comprise one or more sets of reagents for generating a dataset via at least one detection assay that analyzes the test sample from the subject. In various embodiments, the set of reagents enables detection of quantitative values of metabolite biomarkers, such as any of the metabolite biomarkers described herein and in particular, any of the metabolite biomarkers described in Tables 1 or 2.

A kit can include instructions for use of one or more sets of reagents. For example, a kit can include instructions for performing at least one marker quantification assay, examples of which are described herein. In various embodiments, the kits include instructions for practicing the methods disclosed herein (e.g., methods for training or deploying a prediction model to predict risk of cancer). These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### X. Systems

Further disclosed herein are systems for predicting risk of cancer in a subject. In various embodiments, such a system can include one or more sets of reagents for detecting quantitative values of biomarkers in one or more panels of a prediction model, an apparatus configured to receive a mixture of the one or more sets of reagents and a test sample obtained from a subject to measure the quantitative values of the biomarkers, and a computer system communicatively coupled to the apparatus to obtain the measured quantitative values and to implement the prediction model to predict risk of cancer in a subject.

The one or more sets of reagents enable the detection of quantitative levels of the biomarkers in the biomarker panel. In various embodiments, the one or more sets of reagents involve reagents used to perform one or more assays more measuring levels of protein biomarkers and/or metabolites. For example, the reagents include one or more antibodies that bind to one or more of the biomarkers. The antibodies may be monoclonal antibodies or polyclonal antibodies. As another example, the reagents can include reagents for performing ELISA including buffers and detection agents.

The apparatus is configured to detect quantitative levels of biomarkers in a mixture of a reagent and test sample. As an example, the apparatus can determine quantitative levels of biomarkers through a metabolite detection assay (e.g., a metabolite detection assay that uses one of NMR spectroscopy or LC-MS).

The mixture of the reagent and test sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading to generate quantitative values of biomarkers. Examples of an apparatus include a plate reader (e.g., a luminescent plate reader, absorbance plate reader, fluorescence plate reader), a spectrometer, and a spectrophotometer. Further examples of an apparatus include an NMR spectroscopy system or a LC-MS system.

The computer system, such as example computer 400 described in FIG. 4, communicates with the apparatus to receive the quantitative values of biomarkers. The computer system implements, in silico, a prediction model to analyze the quantitative values of the biomarkers and predict risk of cancer for the subject.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should be allowed for.

### Example 1: Study Methods

This study was performed using data and biospecimens collected as part of the Liverpool Lung Project (LLP) cohort, and were obtained following institutional review board approval, and patients provided written informed consent. Leveraging the Liverpool Lung Project (LLP), a unique 10-year observational cohort that followed subjects from healthy to lung cancer diagnoses, pre-diagnosis biomarkers were generated for 181 healthy subjects and 91 lung cancer subjects with samples taken 1-5 years before their diagnosis.

The study was designed to detect ‘predictive’ biomarkers (1027 markers from the Metabolon platform for the detection and quantification of metabolomics) for lung cancer in a healthy population of which one third developed lung cancer during follow-up. The study included a nested-case-control (NCC) design with 92 subjects that developed lung cancer each combined with two matched control subjects based on age, gender and smoking behavior. The total study comprised 92 ‘triplets’ (e.g., 276 subjects in total).

Samples were processed using the HTG Metabolon HD4 Platform workflow. Two-hundred and seventy-six (276) plasma samples were extracted and split into equal parts for analysis on the three Liquid chromatography tandem-mass spectrometry (LC-MS/MS) methods, and a Polar LC method. Ions were matched to an in-house library of standards for metabolite identification and for metabolite quantitation by peak area integration.

### Example 2: Example Algorithm for Training a Prediction Model

Two separate approaches were implemented to predict risk of lung cancer: a binary outcome model through random forest (Example 3) and a time to event model using Cox Elastic Net model (Example 4). AUCs from the models and recursive feature elimination are reported from 5-fold cross validation repeated 5 times. The Cox Elastic Net was developed to explore the relationship between different biomarkers and time to lung cancer development. Biomarkers were initially selected using p values from univariate Cox models. The random forest model was developed as a binary model to predict cancer vs. healthy based on different biomarkers regardless of time to lung cancer development. Biomarkers for the binary model were selected based on differential levels between healthy and cancer subjects (linear model, p<0.05).

For the Cox Elastic Net model, the panels with the most predictive panels of biomarkers for the current lung cancer status were derived by penalized regression techniques using elastic net regulation.

The models were optimized to yield the best prediction of the risk of lung cancer. The simultaneous estimation of the probability of having either type of lung cancer, as being predictable from the biomarker values, was done by the implementation of a modified multinomial approach to the elastic net framework.

The derivation of the best set of tuning parameters of the elastic net was optimized by adding p value information from univariate screening to the optimization process. Inclusion of a threshold on the p value from the univariate screening allowed to exclude large numbers of non-relevant biomarkers, which significantly accelerated the search process and yielded more stable and more reproducible panels of biomarkers. The selection of the best combination of elastic net tuning settings was designed to find the most stable combination of (1) the p value from the univariate screening, (2) the mix of LASSO and Ridge penalization (a) and (3) the overall penalization level (2), using the most stringent penalty within the confidence limits of the lowest cross validation error from a leave-one-out cross validation screening. In order to lower the risk of the false-positive selection of predicting biomarkers with low levels, a penalty factor was added to the general Elastic Net penalty based on the proportion of values of each biomarker at or below the lower limit of quantitation (LLOQ).

### Example 3: Example Panel in a Binary Prediction Model

In this example, a binary prediction model was constructed for predicting presence or absence of cancer based on metabolite biomarker levels. Here, a binary random forest prediction model was constructed by incorporating an initial set of predictors, followed by recursive feature elimination to reduce the total number of predictors in the model.

Here, the binary random forest model was constructed in accordance with the embodiment shown in FIG. 3. Thus, the binary random forest model analyzes biomarker levels and generates a cancer score that is informative for the overall prediction (e.g., presence or absence of cancer).

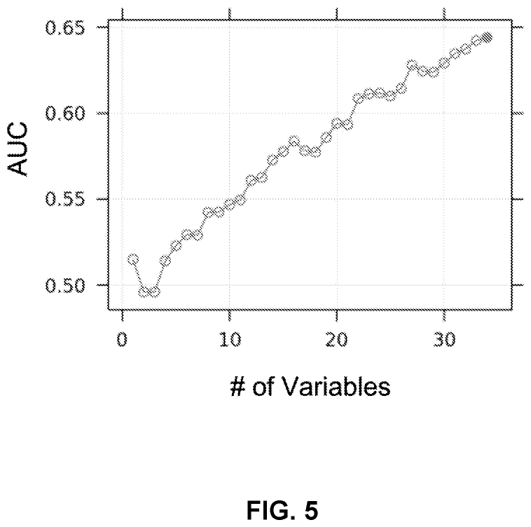

Table 1 below shows the predictors that were included in the binary random forest model. Table 1 further identifies the recursive feature elimination (RFE) rank of each metabolite biomarker. FIG. 5 shows the performance of the binary random forest predictive model as a function of the number of predictors in the model, in accordance with the embodiment of the prediction model shown in FIG. 3. Beginning with the 34 initial metabolite biomarkers (34 biomarkers shown in Table 1), the performance of the binary random forest model was evaluated as metabolite biomarkers were iteratively removed via RFE. For example, with the 34 initial metabolite biomarkers (indicated on the x-axis of FIG. 5 as “variables”), the predictive model achieved an AUC performance metric of nearly 0.65. As the number of metabolite biomarkers decreased, the predictive capacity of the random forest model remained predictive. For example, at 20 metabolite biomarkers (which includes the biomarkers in Table 1 with corresponding RFE rank between 1-20), the random forest predictive model exhibited an AUC of ˜0.60. At 10 metabolite biomarkers (which includes the biomarkers in Table 1 with corresponding RFE rank between 1-10), the random forest predictive model exhibited an AUC of ˜0.55. At 5 metabolite biomarkers (which includes the biomarkers in Table 1 with corresponding RFE rank between 1-5), the random forest predictive model exhibited an AUC of ˜0.53.

TABLE 1
Identification of biomarkers in binary random forest model. “RI”
refers to retention index. “PUBCHEM,” “CAS,” “KEGG,”
and “Group HMDB” refer to the four publicly available databases
in which the metabolite identifier (if present) is cataloged.

| RFE | | | | |
|---|---|---|---|---|
| Rank | BIOCHEMICAL | SUPER_PATHWAY | SUB_PATHWAY | PLATFORM |
| 1 | beta- | Amino Acid | Leucine, Isoleucine | LC/MS |
| | hydroxyisovaleroyl | | and Valine | Pos Early |
| | carnitine | | Metabolism | |
| 2 | pyrraline | Xenobiotics | Food | LC/MS |
| | | | Component/Plant | Pos Early |
| 3 | citramalate | Amino Acid | Glutamate | LC/MS |
| | | | Metabolism | Pos Early |
| 4 | succinate | Energy | TCA Cycle | LC/MS |
| | | | | Polar |
| 5 | urate | Nucleotide | Purine Metabolism, | LC/MS |
| | | | (Hypo)Xanthine/ | Neg |
| | | | Inosine containing | |
| 6 | 2-aminophenol | Xenobiotics | Food | LC/MS |
| | sulfate | | Component/Plant | Neg |
| 7 | guanidinosuccinate | Amino Acid | Guanidino and | LC/MS |
| | | | Acetamido | Polar |
| | | | Metabolism | |
| 8 | docosahexaenoylcholine | Lipid | Fatty Acid | LC/MS |
| | | | Metabolism (Acyl | Pos Late |
| | | | Choline) | |
| 9 | sphingomyelin | Lipid | Sphingomyelins | LC/MS |
| | (d18:2/18:1) | | | Pos Late |
| 10 | homocitrulline | Amino Acid | Urea cycle; | LC/MS |
| | | | Arginine and | Polar |
| | | | Proline Metabolism | |
| 11 | hypotaurine | Amino Acid | Methionine, | LC/MS |
| | | | Cysteine, SAM and | Polar |
| | | | Taurine Metabolism | |
| 12 | allantoin | Nucleotide | Purine Metabolism, | LC/MS |
| | | | (Hypo)Xanthine/ | Polar |
| | | | Inosine containing | |
| 13 | dimethyl sulfone | Xenobiotics | Chemical | LC/MS |
| | | | | Pos Early |
| 14 | N-palmitoyl- | Lipid | Ceramides | LC/MS |
| | sphingosine | | | Pos Late |
| | (d18:1/16:0) | | | |
| 15 | 2-hydroxysebacate | Lipid | Fatty Acid, | LC/MS |
| | | | Dicarboxylate | Neg |
| 16 | N- | Amino Acid | Alanine and | LC/MS |
| | carbamoylalanine | | Aspartate | Pos Early |
| | | | Metabolism | |
| 17 | 3-methoxytyrosine | Amino Acid | Tyrosine | LC/MS |
| | | | Metabolism | Pos Early |
| 18 | 2-palmitoyl-GPC | Lipid | Lysophospholipid | LC/MS |
| | (16:0) | | | Neg |
| 19 | 2-hydroxystearate | Lipid | Fatty Acid, | LC/MS |
| | | | Monohydroxy | Neg |
| 20 | threonine | Amino Acid | Glycine, Serine and | LC/MS |
| | | | Threonine | Pos Early |
| | | | Metabolism | |
| 21 | 3beta-hydroxy-5- | Lipid | Sterol | LC/MS |
| | cholestenoate | | | Neg |
| 22 | lactose | Carbohydrate | Disaccharides and | LC/MS |
| | | | Oligosaccharides | Polar |
| 23 | 2,4-di-tert- | Xenobiotics | Chemical | LC/MS |
| | butylphenol | | | Neg |
| 24 | histidine | Amino Acid | Histidine | LC/MS |
| | | | Metabolism | Neg |
| 25 | 2-palmitoleoyl- | Lipid | Lysophospholipid | LC/MS |
| | GPC (16:1) | | | Neg |
| 26 | alpha-ketoglutarate | Energy | TCA Cycle | LC/MS |
| | | | | Polar |
| 27 | dihomo- | Lipid | Fatty Acid | LC/MS |
| | linolenoylcarnitine | | Metabolism (Acyl | Pos Late |
| | (C20:3n3 or 6) | | Carnitine, | |
| | | | Polyunsaturated) | |
| 28 | arachidonoylcarnitine | Lipid | Fatty Acid | LC/MS |
| | (C20:4) | | Metabolism (Acyl | Pos Late |
| | | | Carnitine, | |
| | | | Polyunsaturated) | |
| 29 | cysteinylglycine | Amino Acid | Glutathione | LC/MS |
| | | | Metabolism | Pos Early |
| 30 | 1-palmitoyl-GPA | Lipid | Lysophospholipid | LC/MS |
| | (16:0) | | | Neg |
| 31 | stearoylcholine | Lipid | Fatty Acid | LC/MS |
| | | | Metabolism (Acyl | Pos Late |
| | | | Choline) | |
| 32 | sulfate of piperine | Xenobiotics | Food | LC/MS |
| | metabolite | | Component/Plant | Neg |
| | C16H19NO3 | | | |
| 33 | cyclo(phe-pro) | Peptide | Dipeptide | LC/MS |
| | | | | Pos Early |
| 34 | salicyluric | Xenobiotics | Drug - Analgesics, | LC/MS |
| | glucuronide | | Anesthetics | Neg |

| RFE | | | | | | |
|---|---|---|---|---|---|---|
| Rank | RI | MASS | PUBCHEM | CAS | KEGG | Group.HMDB |
| 1 | 2584 | 262.1649 | | 99159-87-2 | | |
| 2 | 2750 | 255.1339 | 122228 | 74509-14-1 | | HMDB33143 |
| 3 | 1224 | 166.071 | 1081 | Oct. 8, 1936 | C00815 | HMDB00426 |
| 4 | 3149 | 117.0193 | 1110 | 110-15-6 | C00042 | HMDB00254 |
| 5 | 757.1 | 167.0211 | 1175 | 69-93-2; | C00366 | HMDB00289 |
| | | | | 120K5305 | | |
| 6 | 1677 | 188.0023 | 181670 | | | HMDB61116 |
| 7 | 3550 | 174.052 | 97856 | 6133-30-8 | C03139 | HMDB03157 |
| 8 | 1378 | 414.3367 | 447216268 | | | HMDB0240647 |
| 9 | 2025 | 727.5749 | | | | |
| 10 | 3006.8 | 188.1041 | 65072 | 1190-49-4 | C02427 | HMDB00679 |
| 11 | 2763.4 | 108.0125 | 107812 | 300-84-5 | C00519 | HMDB00965 |
| 12 | 1672 | 157.0367 | 204 | 97-59-6 | C02350 | HMDB00462 |
| 13 | 832 | 95.0161 | 6213 | 67-71-0 | C11142 | HMDB04983 |
| 14 | 2893 | 538.5194 | 5283564 | 24696-26-2 | | HMDB04949 |
| 15 | 1324 | 217.1081 | 128458 | 103963-71-9 | | HMDB00424 |
| 16 | 950 | 133.0608 | 426409 | 77340-50-2 | | |
| 17 | 2555 | 212.0917 | 1670 | 300-48-1 | | HMDB01434 |
| 18 | 6215 | 570.3413 | 15061532 | | | HMDB61702 |
| 19 | 5695 | 299.2592 | 69417 | 629-22-1 | C03045 | |
| 20 | 1514 | 120.0655 | 6288 | 72-19-5 | C00188 | HMDB00167 |
| 21 | 5398 | 415.3218 | 165511 | 6561-58-6 | C17333 | |
| 22 | 3500 | 387.1144 | 84571 | 5965-66-2 | C00243 | HMDB00186 |
| 23 | 5749 | 205.1597 | | 96-76-4 | | |
| 24 | 755.9 | 154.0622 | 6274 | 5934-29-2 | C00135 | HMDB00177 |
| 25 | 5720 | 568.3256 | | | | HMDB10383 |
| 26 | 2700 | 145.0143 | 51 | 305-72-6; | C00026 | HMDB00208 |
| | | | | 328-50-7; | | |
| | | | | 22202-68-2 | | |
| 27 | 1392 | 450.3578 | | | | |
| 28 | 1353 | 448.3422 | | | | HMDB0006455 |
| 29 | 2132 | 179.0485 | 439498 | 19246-18-5 | C01419 | HMDB00078 |
| 30 | 5600 | 409.2361 | 6419701 | 17618-08-5 | C04036 | HMDB00327 |
| 31 | 1520 | 370.368 | | | | HMDB0240648 |
| 32 | 4112 | 352.086 | | | | |
| 33 | 2766 | 245.1285 | 9837725; | 3705-26-8 | C11847 | |
| | | | 443440 | | | |
| 34 | 1463 | 370.078 | 121596205 | | | HMDB0240252 |

### Example 4: Example Panel in a Time to Event Prediction Model

In this example, a prediction model was constructed for predicting risk of cancer within 1-5 years. Here, the prediction model was constructed according to the embodiment shown in FIG. 3. Specifically, an initial Cox Elastic Net model was built incorporating an initial set of predictors, followed by recursive feature elimination to reduce the total number of predictors in the model. A common Cox Elastic net was implemented using p values from univariate stage-independent Cox models as inclusion filter for the predictors.

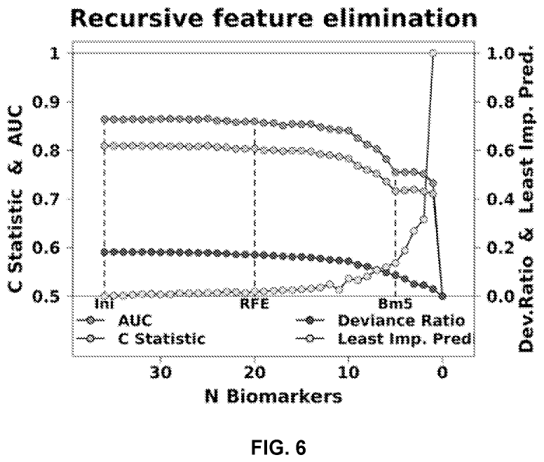

The Cox Elastic net model analyzes biomarker levels and generates a cancer score that is informative for the overall prediction (e.g., likelihood of developing cancer within a particular time period). Table 2 below shows the predictors that were included in the Cox Elastic net model. Table 2 further identifies the recursive feature elimination (RFE) rank of each metabolite biomarker. FIG. 6 shows the performance of a Cox Elastic net predictive model during training as a function of the number of predictors in the model, in accordance with the embodiment of the prediction model shown in FIG. 3. Beginning with the 36 initial metabolite biomarkers (36 biomarkers shown in Table 2), the performance of the Cox Elastic net model was evaluated as metabolite biomarkers were iteratively removed via RFE. For example, with the 36 initial metabolite biomarkers (indicated on the x-axis of FIG. 6 as “N-biomarkers”), the predictive model achieved an AUC performance metric of ˜0.87. As the number of metabolite biomarkers decreased, the predictive capacity of the Cox Elastic net model remained predictive. For example, at 20 metabolite biomarkers (which includes the biomarkers in Table 2 with corresponding RFE rank between 1-20), the Cox Elastic net predictive model exhibited an AUC of ˜0.85 (as shown in FIG. 6). At 10 metabolite biomarkers (which includes the biomarkers in Table 2 with corresponding RFE rank between 1-10), the Cox Elastic net predictive model exhibited an AUC of 0.84. At 5 metabolite biomarkers (which includes the biomarkers in Table 2 with corresponding RFE rank between 1-5), the Cox Elastic net predictive model exhibited an AUC of 0.75.

TABLE 2
Identification of biomarkers in time to event model. “RI” refers
to retention index. “PUBCHEM,” “CAS,” “KEGG,”
and “Group HMDB” refers to the four publicly available databases
in which the metabolite identifier (if present) is cataloged.

| RFE | | | | |
|---|---|---|---|---|
| Rank | BIOCHEMICAL | SUPER_PATHWAY | SUB_PATHWAY | PLATFORM |
| 1 | pseudoephedrine | Xenobiotics | Drug - Respiratory | LC/MS Pos |
| | | | | Early |
| 2 | 3-(cystein-S- | Xenobiotics | Drug - Analgesics, | LC/MS Pos |
| | yl)acetaminophen | | Anesthetics | Early |
| 3 | 2- | Xenobiotics | Drug - Analgesics, | LC/MS Neg |
| | methoxyacetaminophen | | Anesthetics | |
| | sulfate | | | |
| 4 | alliin | Xenobiotics | Food | LC/MS Pos |
| | | | Component/Plant | Early |
| 5 | daidzein sulfate | Xenobiotics | Food | LC/MS Neg |
| | | | Component/Plant | |
| 6 | alpha-ketoglutarate | Energy | TCA Cycle | LC/MS Polar |
| 7 | sedoheptulose | Carbohydrate | Pentose | LC/MS Polar |
| | | | Metabolism | |
| 8 | 1-cerotoyl-GPC | Lipid | Lysophospholipid | LC/MS Pos |
| | (26:0) | | | Late |
| 9 | 3-hydroxy-2- | Xenobiotics | Chemical | LC/MS Neg |
| | methylpyridine | | | |
| | sulfate | | | |
| 10 | cysteine sulfinic | Amino Acid | Methionine, | LC/MS Pos |
| | acid | | Cysteine, SAM | Early |
| | | | and Taurine | |
| | | | Metabolism | |
| 11 | docosahexaenoylcholine | Lipid | Fatty Acid | LC/MS Pos |
| | | | Metabolism (Acyl | Late |
| | | | Choline) | |
| 12 | stearoylcholine | Lipid | Fatty Acid | LC/MS Pos |
| | | | Metabolism (Acyl | Late |
| | | | Choline) | |
| 13 | glucuronide of | Partially | Partially | LC/MS Neg |
| | C10H18O2 | Characterized | Characterized | |
| | | Molecules | Molecules | |
| 14 | N- | Amino Acid | Alanine and | LC/MS Pos |
| | carbamoylalanine | | Aspartate | Early |
| | | | Metabolism | |
| 15 | cyclo(phe-pro) | Peptide | Dipeptide | LC/MS Pos |
| | | | | Early |
| 16 | 4-acetamidophenol | Xenobiotics | Drug - Analgesics, | LC/MS Neg |
| | | | Anesthetics | |
| 17 | allantoin | Nucleotide | Purine | LC/MS Polar |
| | | | Metabolism, | |
| | | | (Hypo)Xanthine/ | |
| | | | Inosine containing | |
| 18 | salicyluric | Xenobiotics | Drug - Analgesics, | LC/MS Neg |
| | glucuronide | | Anesthetics | |
| 19 | pyrraline | Xenobiotics | Food | LC/MS Pos |
| | | | Component/Plant | Early |
| 20 | 3-hydroxycotinine | Xenobiotics | Tobacco | LC/MS Neg |
| | glucuronide | | Metabolite | |
| 21 | 2,4-di-tert- | Xenobiotics | Chemical | LC/MS Neg |
| | butylphenol | | | |
| 22 | 2-palmitoyl-GPC | Lipid | Lysophospholipid | LC/MS Neg |
| | (16:0) | | | |
| 23 | succinate | Energy | TCA Cycle | LC/MS Polar |
| 24 | 2-aminophenol | Xenobiotics | Food | LC/MS Neg |
| | sulfate | | Component/Plant | |
| 25 | 1-palmitoleoyl-2- | Lipid | Phosphatidylcholine | LC/MS Pos |
| | linolenoyl-GPC | | (PC) | Late |
| | (16:1/18:3) | | | |
| 26 | N-(2- | Xenobiotics | Food | LC/MS Pos |
| | furoyl)glycine | | Component/Plant | Early |
| 27 | 3beta-hydroxy-5- | Lipid | Sterol | LC/MS Neg |
| | cholestenoate | | | |
| 28 | guanidinosuccinate | Amino Acid | Guanidino and | LC/MS Polar |
| | | | Acetamido | |
| | | | Metabolism | |
| 29 | gamma- | Peptide | Gamma-glutamyl | LC/MS Pos |
| | glutamylhistidine | | Amino Acid | Early |
| 30 | citramalate | Amino Acid | Glutamate | LC/MS Pos |
| | | | Metabolism | Early |
| 31 | 2-hydroxysebacate | Lipid | Fatty Acid, | LC/MS Neg |
| | | | Dicarboxylate | |
| 32 | 2- | Xenobiotics | Drug - Analgesics, | LC/MS Neg |
| | methoxyacetaminophen | | Anesthetics | |
| | glucuronide | | | |
| 33 | urate | Nucleotide | Purine | LC/MS Neg |
| | | | Metabolism, | |
| | | | (Hypo)Xanthine/ | |
| | | | Inosine containing | |
| 34 | hypotaurine | Amino Acid | Methionine, | LC/MS Polar |
| | | | Cysteine, SAM | |
| | | | and Taurine | |
| | | | Metabolism | |
| 35 | 5alpha-androstan- | Lipid | Androgenic | LC/MS Neg |
| | 3alpha,17beta-diol | | Steroids | |
| | monosulfate | | | |
| 36 | homocitrulline | Amino Acid | Urea cycle; | LC/MS Polar |
| | | | Arginine and | |
| | | | Proline | |
| | | | Metabolism | |

| RFE | | | | | | |
|---|---|---|---|---|---|---|
| Rank | RI | MASS | PUBCHEM | CAS | KEGG | Group.HMDB |
| 1 | 3041 | 166.1226 | 7028 | 90-82-4 | C02765 | HMDB01943 |
| 2 | 2420 | 271.0747 | 5233914 | | | |
| 3 | 1949 | 260.0234 | 86290014 | | | |
| 4 | 1470 | 178.0532 | 87310 | 556-27-4 | C08265 | HMDB33592 |
| 5 | 3330 | 333.0076 | 129661094 | | | HMDB0242171 |
| 6 | 2700 | 145.0143 | 51 | 305-72-6; | C00026 | HMDB00208 |
| | | | | 328-50-7; | | |
| | | | | 22202-68-2 | | |
| 7 | 2255 | 209.0667 | 5459879 | 3019-74-7 | | HMDB03219 |
| 8 | 1990 | 636.4963 | | | | HMDB29205 |
| 9 | 1849 | 188.0023 | | | | |
| 10 | 597 | 154.0169 | 109 | 207121-48-0 | C00606 | HMDB00996 |
| 11 | 1378 | 414.3367 | 447216268 | | | HMDB0240647 |
| 12 | 1520 | 370.368 | | | | HMDB0240648 |
| 13 | 2728 | 345.1555 | | | | |
| 14 | 950 | 133.0608 | 426409 | 77340-50-2 | | |
| 15 | 2766 | 245.1285 | 9837725; | 3705-26-8 | C11847 | |
| | | | 443440 | | | |
| 16 | 2174 | 150.0561 | 1983 | 103-90-2 | C06804 | HMDB01859 |
| 17 | 1672 | 157.0367 | 204 | 97-59-6 | C02350 | HMDB00462 |
| 18 | 1463 | 370.078 | 121596205 | | | HMDB0240252 |
| 19 | 2750 | 255.1339 | 122228 | 74509-14-1 | | HMDB33143 |
| 20 | 1745 | 367.1147 | 183115 | 132929-88-5 | | HMDB01204 |
| 21 | 5749 | 205.1597 | 7311 | 96-76-4 | | HMDB0013816 |
| 22 | 6215 | 570.3413 | 15061532 | | | HMDB61702 |
| 23 | 3149 | 117.0193 | 1110 | 110-15-6 | C00042 | HMDB00254 |
| 24 | 1677 | 188.0023 | 181670 | | | HMDB61116 |
| 25 | 1900 | 754.5381 | | | | HMDB08008 |
| 26 | 1600 | 170.0448 | 21863 | 5657-19-2; | | HMDB00439 |
| | | | | 5657-19-2 | | |
| 27 | 5398 | 415.3218 | 165511 | 6561-58-6 | C17333 | |
| 28 | 3550 | 174.052 | 97856 | 6133-30-8 | C03139 | HMDB03157 |
| 29 | 2740 | 285.1194 | 7017195 | 37460-15-4 | | |
| 30 | 1224 | 166.071 | 1081 | Oct. 8, 1936 | C00815 | HMDB00426 |
| 31 | 1324 | 217.1081 | 128458 | 103963-71-9 | | HMDB00424 |
| 32 | 1633 | 356.0987 | 14367271 | | | |
| 33 | 757.1 | 167.0211 | 1175 | 69-93-2; | C00366 | HMDB00289 |
| | | | | 120K5305 | | |
| 34 | 2763 | 108.0125 | 107812 | 300-84-5 | C00519 | HMDB00965 |
| 35 | 5080 | 371.1898 | | | | |
| 36 | 3007 | 188.1041 | 65072 | 1190-49-4 | C02427 | HMDB00679 |

1. A method for predicting risk of cancer in a subject, the method comprising:
obtaining or having obtained a dataset comprising quantitative levels of a plurality of biomarkers, wherein the plurality of biomarkers comprises metabolite biomarkers comprising two or more of Beta-hydroxyisovaleroylcarnitine, Pyrraline, Citramalate, Succinate, and Urate, and
generating a prediction of risk of cancer for the subject by applying a predictive model to the quantitative values of the plurality of biomarkers.

2. The method of claim 1, wherein the metabolite biomarkers comprise three or more of Beta-hydroxyisovaleroylcarnitine, Pyrraline, Citramalate, Succinate, and Urate.

3. The method of claim 1, wherein the metabolite biomarkers comprise four or more of Beta-hydroxyisovaleroylcarnitine, Pyrraline, Citramalate, Succinate, and Urate.

4. The method of claim 1, wherein the metabolite biomarkers comprise each of Beta-hydroxyisovaleroylcarnitine, Pyrraline, Citramalate, Succinate, and Urate.

5. The method of any one of claims 1-4, wherein the metabolite biomarkers further comprise one or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

6. The method of any one of claims 1-4, wherein the metabolite biomarkers further comprise five or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

7. The method of any one of claims 1-4, wherein the metabolite biomarkers further comprise ten or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

8. The method of any one of claims 1-4, wherein the metabolite biomarkers further comprise each of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

9. The method of any one of claims 1-8, wherein the metabolite biomarkers further comprise one or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

10. The method of any one of claims 1-8, wherein the metabolite biomarkers further comprise five or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

11. The method of any one of claims 1-8, wherein the metabolite biomarkers further comprise ten or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

12. The method of any one of claims 1-8, wherein the metabolite biomarkers further comprise each of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

13. A method for predicting risk of cancer in a subject, the method comprising:
obtaining or having obtained a dataset comprising quantitative levels of a plurality of biomarkers, wherein the plurality of biomarkers comprises metabolite biomarkers comprising two or more of pseudoephedrine, 3-(cystein-S-yl) acetaminophen, 2-methoxyacetaminophen sulfate, alliin, and daidzein sulfate, and
generating a prediction of risk of cancer for the subject by applying a predictive model to the quantitative values of the plurality of biomarkers.

14. The method of claim 13, wherein the metabolite biomarkers comprise three or more of pseudoephedrine, 3-(cystein-S-yl) acetaminophen, 2-methoxyacetaminophen sulfate, alliin, and daidzein sulfate.

15. The method of claim 13, wherein the metabolite biomarkers comprise four or more of pseudoephedrine, 3-(cystein-S-yl) acetaminophen, 2-methoxyacetaminophen sulfate, alliin, and daidzein sulfate.

16. The method of claim 13, wherein the metabolite biomarkers comprise each of pseudoephedrine, 3-(cystein-S-yl) acetaminophen, 2-methoxyacetaminophen sulfate, alliin, and daidzein sulfate.

17. The method of any one of claims 13-16, wherein the metabolite biomarkers further comprise one or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

18. The method of any one of claims 13-16, wherein the metabolite biomarkers further comprise five or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

19. The method of any one of claims 13-16, wherein the metabolite biomarkers further comprise ten or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

20. The method of any one of claims 13-16, wherein the metabolite biomarkers further comprise each of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

21. The method of any one of claims 13-20, wherein the metabolite biomarkers further comprise one or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

22. The method of any one of claims 13-20, wherein the metabolite biomarkers further comprise five or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

23. The method of any one of claims 13-20, wherein the metabolite biomarkers further comprise ten or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

24. The method of any one of claims 13-20, wherein the metabolite biomarkers further comprise each of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

25. The method of any one of claims 1-24, wherein the cancer is lung cancer.

26. The method of any one of claims 1-25, wherein the risk of cancer is a level of risk of the subject developing cancer within 1 year, within 2 years, within 3 years, within 4 years, within 5 years, within 6 years, within 7 years, within 8 years, within 9 years, or within 10 years.

27. The method of any one of claims 1-25, wherein the risk of cancer is a presence or absence of cancer.

28. The method of claim 26, wherein the level of risk is one of a low risk, medium risk, or high risk.

29. The method of any one of claims 1-28, wherein the dataset is derived from a test sample obtained from the subject.

30. The method of claim 29, wherein the test sample is a blood or serum sample.

31. The method of any one of claims 1-30, wherein obtaining or having obtained the dataset comprises performing one or more assays.

32. The method of claim 31, wherein performing the one or more assays comprises performing one or more of liquid chromatography (LC), gas chromatography (GC) (e.g., GC using an electron capture detector), a nitrogen/phosphorous detector, a flame photometric detector, high performance liquid chromatography (HPLC), nuclear magnetic resonance (NMR), mass spectrometry (MS), liquid chromatography MS (LC-MS), high performance LC-MS (HPLC-MS), or ultrahigh performance liquid chromatography-tandem MS (UPLC-MS/MS).

33. The method of any one of claims 1-32, further comprising:
selecting a therapy for providing to the subject based on the prediction of cancer.

34. A non-transitory computer readable medium comprising instructions that, when executed by a processor, cause the processor to:
obtain or have obtained a dataset comprising quantitative levels of a plurality of biomarkers, wherein the plurality of biomarkers comprises metabolite biomarkers comprising two or more of Beta-hydroxyisovaleroylcarnitine, Pyrraline, Citramalate, Succinate, and Urate, and
generate a prediction of risk of cancer for the subject by applying a predictive model to the quantitative values of the plurality of biomarkers.

35. The non-transitory computer readable medium of claim 34, wherein the metabolite biomarkers comprise three or more of Beta-hydroxyisovaleroylcarnitine, Pyrraline, Citramalate, Succinate, and Urate.

36. The non-transitory computer readable medium of claim 34, wherein the metabolite biomarkers comprise four or more of Beta-hydroxyisovaleroylcarnitine, Pyrraline, Citramalate, Succinate, and Urate.

37. The non-transitory computer readable medium of claim 34, wherein the metabolite biomarkers comprise each of Beta-hydroxyisovaleroylcarnitine, Pyrraline, Citramalate, Succinate, and Urate.

38. The non-transitory computer readable medium of any one of claims 34-37, wherein the metabolite biomarkers further comprise one or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

39. The non-transitory computer readable medium of any one of claims 34-37, wherein the metabolite biomarkers further comprise five or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

40. The non-transitory computer readable medium of any one of claims 34-37, wherein the metabolite biomarkers further comprise ten or more of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

41. The non-transitory computer readable medium of any one of claims 34-37, wherein the metabolite biomarkers further comprise each of 2-aminophenol sulfate, guanidinosuccinate, docosahexaenoylcholine, sphingomyelin (d18:2/18:1), homocitrulline, hypotaurine, allantoin, dimethyl sulfone, N-palmitoyl-sphingosine (d18:1/16:0), 2-hydroxysebacate, N-carbamoylalanine, 3-methoxytyrosine, 2-palmitoyl-GPC (16:0), 2-hydroxystearate, and threonine.

42. The non-transitory computer readable medium of any one of claims 34-41, wherein the metabolite biomarkers further comprise one or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

43. The non-transitory computer readable medium of any one of claims 34-41, wherein the metabolite biomarkers further comprise five or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

44. The non-transitory computer readable medium of any one of claims 34-41, wherein the metabolite biomarkers further comprise ten or more of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

45. The non-transitory computer readable medium of any one of claims 34-41, wherein the metabolite biomarkers further comprise each of 3beta-hydroxy-5-cholestenoate, lactose, 2,4-di-tert-butylphenol, histidine, 2-palmitoleoyl-GPC (16:1), alpha-ketoglutarate, dihomo-linolenoylcarnitine (C20:3n3 or 6), arachidonoylcarnitine (C20:4), cysteinylglycine, 1-palmitoyl-GPA (16:0), stearoylcholine, sulfate of piperine metabolite C16H19NO3, cyclo (phe-pro), or salicyluric glucuronide.

46. A non-transitory computer readable medium comprising instructions that, when executed by a processor, cause the processor to:
obtain or have obtained a dataset comprising quantitative levels of a plurality of biomarkers, wherein the plurality of biomarkers comprises metabolite biomarkers comprising two or more of pseudoephedrine, 3-(cystein-S-yl) acetaminophen, 2-methoxyacetaminophen sulfate, alliin, and daidzein sulfate, and
generate a prediction of risk of cancer for the subject by applying a predictive model to the quantitative values of the plurality of biomarkers.

47. The non-transitory computer readable medium of claim 46, wherein the metabolite biomarkers comprise three or more of pseudoephedrine, 3-(cystein-S-yl) acetaminophen, 2-methoxyacetaminophen sulfate, alliin, and daidzein sulfate.

48. The non-transitory computer readable medium of claim 46, wherein the metabolite biomarkers comprise four or more of pseudoephedrine, 3-(cystein-S-yl) acetaminophen, 2-methoxyacetaminophen sulfate, alliin, and daidzein sulfate.

49. The non-transitory computer readable medium of claim 46, wherein the metabolite biomarkers comprise each of pseudoephedrine, 3-(cystein-S-yl) acetaminophen, 2-methoxyacetaminophen sulfate, alliin, and daidzein sulfate.

50. The non-transitory computer readable medium of any one of claims 46-49, wherein the metabolite biomarkers further comprise one or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

51. The non-transitory computer readable medium of any one of claims 46-49, wherein the metabolite biomarkers further comprise five or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

52. The non-transitory computer readable medium of any one of claims 46-49, wherein the metabolite biomarkers further comprise ten or more of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

53. The non-transitory computer readable medium of any one of claims 46-49, wherein the metabolite biomarkers further comprise each of alpha-ketoglutarate, sedoheptulose, 1-cerotoyl-GPC (26:0), 3-hydroxy-2-methylpyridine sulfate, cysteine sulfinic acid, docosahexaenoylcholine, Stearoylcholine, glucuronide of C10H18O2, N-carbamoylalanine, cyclo (phe-pro), 4-acetamidophenol, allantoin, salicyluric glucuronide, pyrraline, and 3-hydroxycotinine glucuronide.

54. The non-transitory computer readable medium of any one of claims 46-53, wherein the metabolite biomarkers further comprise one or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3 alpha, 17beta-diol monosulfate, and homocitrulline.

55. The non-transitory computer readable medium of any one of claims 46-53, wherein the metabolite biomarkers further comprise five or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3 alpha, 17beta-diol monosulfate, and homocitrulline.

56. The non-transitory computer readable medium of any one of claims 46-53, wherein the metabolite biomarkers further comprise ten or more of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3alpha, 17beta-diol monosulfate, and homocitrulline.

57. The non-transitory computer readable medium of any one of claims 46-53, wherein the metabolite biomarkers further comprise each of 2,4-di-tert-butylphenol, 2-palmitoyl-GPC (16:0), succinate, 2-aminophenol sulfate, 1-palmitoleoyl-2-linolenoyl-GPC (16:1/18:3), N-(2-furoyl)glycine, 3beta-hydroxy-5-cholestenoate, guanidinosuccinate, gamma-glutamylhistidine, citramalate, 2-hydroxysebacate, 2-methoxyacetaminophen glucuronide, urate, hypotaurine, 5alpha-androstan-3 alpha, 17beta-diol monosulfate, and homocitrulline.

58. The non-transitory computer readable medium of any one of claims 34-57, wherein the cancer is lung cancer.

59. The non-transitory computer readable medium of any one of claims 34-58, wherein the risk of cancer is a level of risk of the subject developing cancer within 1 year, within 2 years, within 3 years, within 4 years, within 5 years, within 6 years, within 7 years, within 8 years, within 9 years, or within 10 years.

60. The non-transitory computer readable medium of any one of claims 34-58, wherein the risk of cancer is a presence or absence of cancer.

61. The non-transitory computer readable medium of claim 59, wherein the level of risk is one of a low risk, medium risk, or high risk.

62. The non-transitory computer readable medium of any one of claims 34-61, wherein the dataset is derived from a test sample obtained from the subject.

63. The non-transitory computer readable medium of claim 62, wherein the test sample is a blood or serum sample.

64. The non-transitory computer readable medium of any one of claims 34-63, wherein the dataset is obtained from having performed one or more assays.

65. The non-transitory computer readable medium of claim 64, wherein the one or more assays comprises one or more of liquid chromatography (LC), gas chromatography (GC) (e.g., GC using an electron capture detector), a nitrogen/phosphorous detector, a flame photometric detector, high performance liquid chromatography (HPLC), nuclear magnetic resonance (NMR), mass spectrometry (MS), liquid chromatography MS (LC-MS), high performance LC-MS (HPLC-MS), or ultrahigh performance liquid chromatography-tandem MS (UPLC-MS/MS).

66. The method of any of claims 1-33, wherein the prediction model comprises a trained prediction model including one or more panels, each including one or more biomarkers.

67. The method of claim 66, wherein generating the prediction of the risk of cancer for the subject comprises, for each of the one or more panels, outputting a prediction based on the one or more biomarkers of the one or more panels.

68. The method of claim 67, wherein an output prediction of each of the one or more panels is a score.

69. The method of claim 68, wherein generating the prediction of the risk of cancer for the subject comprises combining the scores outputted by the one or more panels to generate an overall prediction.

70. The method of claim 68, wherein generating the prediction of the risk of cancer for the subject comprises generating an overall prediction based on a comparison between a score and one or more reference scores.

71. The non-transitory computer readable medium of any of claims 34-65, wherein the instructions, when executed by a processor, further cause the processor to execute the steps of any of claims 66-70.

72. A computer program comprising instructions which, when the program is executed by a computer, cause the computer to carry out the method of any of claims 1-33 and 66-70.
